(19) United States
(12) Patent Application Publication
(10) Pub. No.: US 2022/0344029 A1
(43) Pub. Date: Oct. 27, 2022

(54) NOVEL SYSTEM AND INFORMATION PROCESSING METHOD FOR ADVANCED NEURO REHABILITATION

(71) Applicant: PRS NEUROSCIENCES & MECHATRONICS RESEARCH INSTITUTE PRIVATE LIMITED, Bangalore (IN)

(72) Inventors: SHARAN SRINIVASAN, BANGALORE (IN); Prathiba Sharan, BANGALORE (IN); Siddharth V, BANGALORE (IN); Ganesh Nagarajan, BANGALORE (IN); Sai Sasanka Jadda, BANGALORE (IN)

(21) Appl. No.: 17/763,736

(22) PCT Filed: Sep. 24, 2020
(86) PCT No.: PCT/IN2020/050814

§ 371 Date: Mar. 25, 2022

(30) Foreign Application Priority Data
Sep. 25, 2019 (IN) 201941038618

(51) Int. Cl. G16H 20/70

(52) U.S. Cl. CPC G16H 20/70

(57) ABSTRACT

A system and an information processing method for advanced neuro rehabilitation is provided. The system comprises processor (201) that may configured to receive a user input that corresponds to a set of answers associated with at least one medical condition of a user to a set of questions associated with the at least one medical condition of the user thorough an input unit. The processor (201) may be further configured to determine objective assessment of at least one of the plurality of BIR stages and the plurality of SCIR stages based on at least one of the first set of data and the clinical evaluation data which is obtained through a clinical evaluation of user. The processor (201) may be further configured to generate, based on the objective assessment, a rehabilitation index that indicates a median probability of functional improvements possible for the user.

### FIELD OF INVENTION

The present invention generally relates to an information processing apparatus and information processing method for advanced neuro rehabilitation.

### BACKGROUND OF THE INVENTION

Neurological disorders are major causes of disability and death across the world. Advancements in neurology and neurosurgery have been relatively successful in reducing mortality. However, the disability aspect of neurological disorders needs more attention from clinicians, researchers and policy makers. Disability due to neurological disorders place a huge clinical, psychological, social and economic burden on affected individuals, their families and the health care system as a whole. Thus, addressing neurological disability is an important health care priority.

The “ought to be” endpoint of clinical neurology is “alive and kicking” rather than “alive and non-functional”.

The science of prediction always has a real-life utilitarian value. Before treatment initiation, patients want to know the odds of treatment success. Clinicians have to be sure-footed with probabilities of good outcomes.

A lot of innovative clinical and technology-driven rehabilitation processes to achieve coma-to-community (C2C)” and “cot-to-community” are currently still in development and patents will be sought later for these novel ideas that are currently in development.

### OBJECT OF THE INVENTION

The principal object of the invention is the capability to generate and provide clear and objective assessment reports on the levels of functioning (and the type of disabilities) of every neurologically disabled patient by introducing a concept of C2C levels—C2C BIR (Brain Injury rehabilitation)—called coma to community and C2C SCIR (Spinal Cord Injury rehabilitation)—called cot-to-community.

Another object of the invention is to predict the progress & prognosis (PPP) of patients from a specific, initial C2C level (irC2C) to any higher C2C level or the best possible C2C level attainable by a patient (exit or erC2C). This is done by computing the Rehabability Index (RI).

The principle of CAREPa-Re®, which helps in bidirectionally connecting clinical, anatomical, radiological and etiopathological correlates of brain and spinal cord/peripheral nerve injuries with assessments of disability and optimal rehabilitation strategies.

Another object of the invention is to provide the rehab professionals with a crystal-clear path and plan of action (that is standardised and person agnostic and hence replicable across the world) which can then be easily explained to the patient and his/her caregivers (by relating it to their day-to-day lives and not some complicated medical jargons)—hence simplifying the communications and also reducing the stress.

These and other objects and characteristics of the present invention will become apparent from the further disclosure to be made in the detailed description given below.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

The invention provides system and information processing method for advanced neuro rehabilitation. The system comprises of a ‘processor’ that has been configured to receive a user input that corresponds to a set of answers associated with at least one medical/neurological condition of a user that is then coded into the functional abilities & disabilities of the user by a set of questions associated with that kind of medical/neurological condition of the user thorough a input unit. The processor may further configured to determine objective assessment of at least one of the plurality of BIR stages and the plurality of SCIR stages based on at least one of the first set of data and the clinical evaluation data which is obtained through a clinical evaluation of user. The processor has been further configured to generate, based on the objective assessment, a detailed individual report and a rehabilitation index (RI) that indicates a median probability of functional improvements possible for the user.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF DRAWINGS

The foregoing and other features of embodiments will become more apparent from the following detailed description of embodiments when read in conjunction with the accompanying drawings. In the drawings, like reference numerals refer to like elements.

In the following description, for the purposes of explanation, numerous specific details are set forth in order to provide a thorough understanding of the embodiments of the invention. It is apparent, however, to one skilled in the art that the embodiments of the invention may be practiced without these specific details or with an equivalent arrangement. In other instances, well-known structures and devices are shown in block diagram form in order to avoid unnecessarily obscuring the embodiments of the invention. Throughout the disclosure the system and the system to assist teaching via digital classroom, may interchangeably be used.

FIG. 1 illustrates network environment, for enabling advanced neuro rehabilitation, according to one embodiment of the invention.

FIG. 2 illustrates a block diagram of a system for advanced neuro rehabilitation, in accordance with an example embodiment of the present disclosure.

FIG. 3 illustrates an example scenario, with a plurality of coma to community (C2C) levels associated with brain injuries for enabling advanced neuro rehabilitation, according to one embodiment of the invention.

FIG. 4 illustrates an example scenario, with a plurality of cot to community (C2C) for Spinal cord/peripheral nerve Injuries (C2C SCIR) for enabling advanced neuro rehabilitation, according to one embodiment of the invention.

FIGS. 5A and 5B illustrates an example scenario; the NewRo model for rehabilitation of spinal cord/peripheral nerve injuries is called “Cot to Community (C2C)”, according to one embodiment of the invention.

FIG. 6 illustrates an information processing method for enabling advanced neuro rehabilitation, according to one embodiment of the invention.

FIG. 7 illustrates generating a rehabilitation index (RI) for enabling advanced neuro rehabilitation, according to one embodiment of the invention.

FIGS. 8A and 8B illustrate an example scenario, showing case example based on coma to community (C2C) levels associated with brain injuries for enabling advanced neuro rehabilitation, according to one embodiment of the invention.

### DETAILED DESCRIPTION OF INVENTION

The embodiments herein and the various features and advantageous details thereof are explained more fully with reference to the non-limiting embodiments that are illustrated in the accompanying drawings and/or detailed in the following description. Descriptions of well-known components and processing techniques are omitted so as to not unnecessarily obscure the embodiments herein. The examples used herein are intended merely to facilitate an understanding of ways in which the embodiments herein may be practised and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearance of the phrase “in an embodiment” in various places in the specification are not necessarily all referring to the same embodiment, nor are separate or alternative embodiments mutually exclusive of other embodiments. Moreover, various features are described which may be exhibited by som\e embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon the present disclosure.

As used in the application, the term ‘circuitry’ or ‘circuit’ refers to all of the following: (a) hardware-only circuit implementations (such as implementations in only analog and/or digital circuitry) and (b) to combinations of circuits and software (and/or firmware), such as (as applicable): (i) to a combination of processor(s) or (ii) to portions of processor(s)/software (including digital signal processor(s)), software, and memory(ies) that work together to cause an apparatus, such as a mobile phone or server, to perform various functions) and (c) to circuits, such as a microprocessor(s) or a portion of a microprocessor(s), that require software or firmware for operation, even if the software or firmware is not physically present and d) the interconnected brain and spinal cord circuits (both anatomical and functional circuits).

This definition of ‘circuitry’ applies to all uses of this term in this application, including in any claims. As a further example, as used in this application, the term “circuitry” would also cover an implementation of merely a processor (or multiple processors) or portion of a processor and its (or their) accompanying software and/or firmware. The term “circuitry” would also cover, for example and if applicable to the particular claim element, a baseband integrated circuit or applications processor integrated circuit for a mobile phone or a similar integrated circuit in server, a cellular network device, or other network device.

The word “exemplary” is used herein to mean “serving as an example, instance, or illustration.” Any aspect described herein as “exemplary” is not necessarily to be construed as preferred or advantageous over other aspects.

In this description, the term “application” may also include files having executable content, such as: object code, scripts, byte code, markup language files, and patches. In addition, an “application” referred to herein, may also include files that are not executable in nature, such as documents that may need to be opened or other data files that need to be accessed.

The term “content” may also include files having executable content, such as: object code, scripts, byte code, markup language files, and patches. In addition, “content” referred to herein, may also include files that are not executable in nature, such as documents that may need to be opened or other data files that need to be accessed.

As used in this description, the terms “component,” “database,” “module,” “system,” and the like are intended to refer to a computer-related entity, either hardware, firmware, a combination of hardware and software, software, or software in execution. For example, a component may be, but is not limited to being, a process running on a processor, a processor, an object, an executable, a thread of execution, a program, and/or a computer. By way of illustration, both an application running on a computing device and the computing device may be a component. One or more components may reside within a process and/or thread of execution, and a component may be localized on one computer and/or distributed between two or more computers. In addition, these components may execute from various computer readable media having various data structures stored thereon. The components may communicate by way of local and/or remote processes such as in accordance with a signal having one or more data packets (e.g., data from one component interacting with another component in a local system, distributed system, and/or across a network such as the Internet with other systems by way of the signal).

In this description, the terms “communication device,” “wireless device,” “wireless telephone,” “wireless communication device,” and “wireless handset” are used interchangeably. With the advent of third generation (“3G”) wireless technology and four generation (“4G”), greater bandwidth availability has enabled more portable computing devices with a greater variety of wireless capabilities. Therefore, a portable computing device may include a cellular telephone, a pager, a PDA, or wearable device, a smartphone, a navigation device, or a hand-held computer with a wireless connection or link.

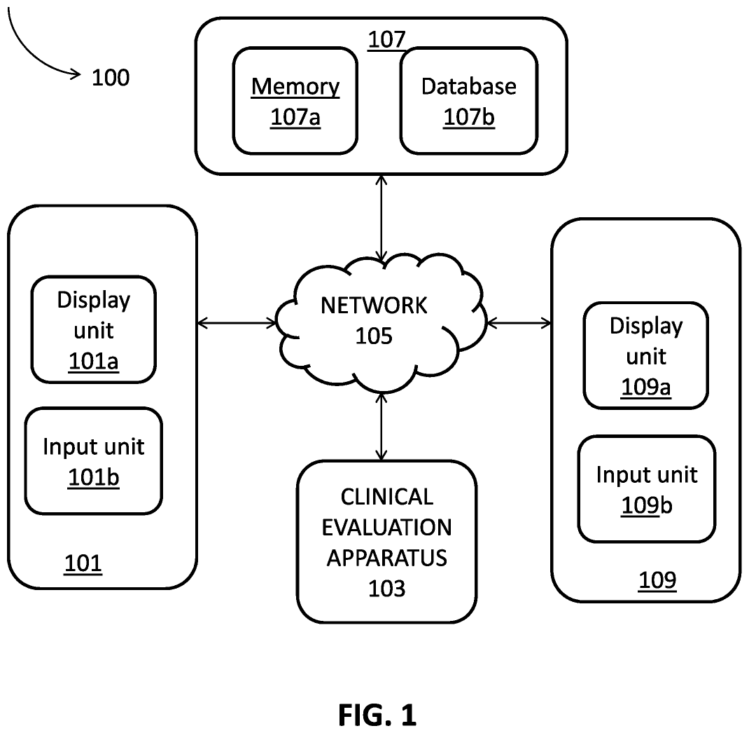

FIG. 1 illustrates network environment, for enabling advanced neuro rehabilitation, according to one embodiment of the invention. Referring now to the drawings, the environment 100 may include a portable computing device 101 associated with a patient or caretaker (hereinafter “first portable device”), a portable device 109 associated with a clinician or rehabilitation professional or a doctor (hereinafter “second portable device”), a server 107, clinical evaluation apparatus 103 and network 105. The first portable device 101 may communicate with a second portable device 109 and also the clinical evaluation apparatus 103, a server 107 via a network 105. The second portable device 109 may be configured to receive instructions explicitly from the local clinical evaluation apparatus 103 and the server through network 107.

The network 105 may include the Internet or any other network capable of communicating data between devices. Suitable networks may include or interface with any one or more of, for instance, a local intranet, a PAN (Personal Area Network), a LAN (Local Area Network), a WAN (Wide Area Network), a MAN (Metropolitan Area Network), a virtual private network (VPN), a storage area network (SAN), a frame relay connection, an Advanced Intelligent Network (AIN) connection, a synchronous optical network (SONET) connection, a digital T1, T3, E1 or E3 line, Digital Data Service (DDS) connection, DSL (Digital Subscriber Line) connection, an Ethernet connection, an ISDN (Integrated Services Digital Network) line, a dial-up port such as a V.90, V.34 or V.34bis analog modem connection, a cable modem, an ATM (Asynchronous Transfer Mode) connection, or an FDDI (Fiber Distributed Data Interface) or CDDI (Copper Distributed Data Interface) connection. Furthermore, communications may also include links to any of a variety of wireless networks, including WAP (Wireless Application Protocol), GPRS (General Packet Radio Service), GSM (Global System for Mobile Communication), CDMA (Code Division Multiple Access) or TDMA (Time Division Multiple Access), cellular phone networks, GPS (Global Positioning System), CDPD (cellular digital packet data), RIM (Research in Motion, Limited) duplex paging network, Bluetooth radio, or an IEEE 802.11-based radio frequency network. The network 105 can further include or interface with any one or more of an RS-232 serial connection, an IEEE-1394 (Firewire) connection, a Fiber Channel connection, an IrDA (infrared) port, a SCSI (Small Computer Systems Interface) connection, a Universal Serial Bus (USB) connection or other wired or wireless, digital or analog interface or connection, mesh or Digi® networking.

In an alternative embodiment, dedicated hardware implementations, such as application specific integrated circuits, programmable logic arrays and other hardware devices, can be constructed to implement one or more of the methods described herein. Applications that may include the apparatus of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The first portable device 101 and a second portable device 109 further comprise an input unit 101b and a display unit 101a. Through the input unit 101b of the first potable device 101 a patient or a caretaker may answer the plurality of questions that is presented in the display unit 101a. The input unit 101b may include buttons or keys, mice, trackballs, touchpads, joysticks. Further, the input unit 101b may include a microphone to receive voice input from the patient or user.

The first portable device 101 according to the present embodiment is an example of an input apparatus to input information by contacting and pressing to the display 101a. The first portable device 101 may be a portable terminal such as a cellular phone, a portable music player and a personal digital assistant (PDA). Further, the mobile device 101 may be an information processing device such as a note type personal computer (PC) and a desktop type PC.

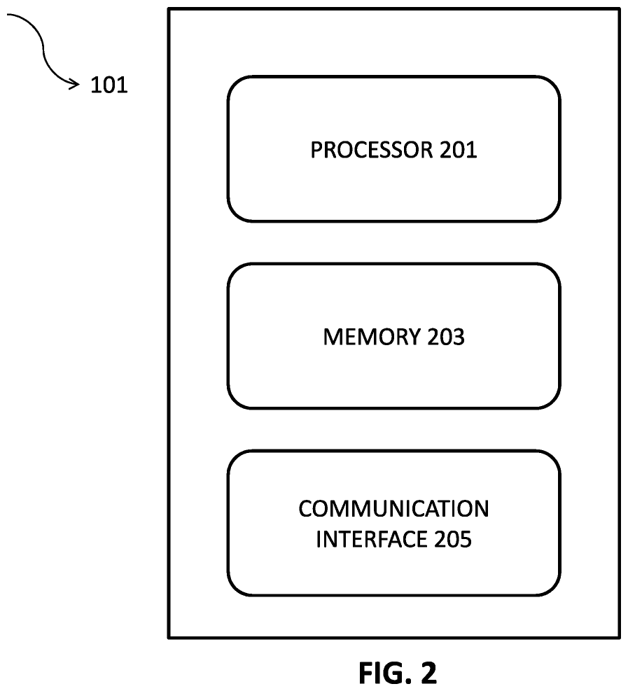

FIG. 2 illustrates a block diagram of system 101 for enabling advanced neuro rehabilitation, according to one embodiment of the invention. To execute activities associated with advanced neuro rehabilitation, the system 101 has embedded a processor 201, a memory 203 and a communication interface 205.

In accordance with an embodiment, the processor 201 may be of any type of processor, such as 32-bit processors using a flat address space, such as a Hitachi SH1, an Intel 80386, an Intel 960, a Motorola 68020 (or other processors having similar or greater addressing space). Processor types other than these, as well as processors that may be developed in the future, are also suitable. The processor may include general processor, Digital Signal Processing (DSP) chip, an Application Specific Integrated Circuit (ASIC), Field Programmable Gate Arrays (FPGAs), AT89S52 microcontroller firmware or a combination thereof.

Processors suitable for the execution of a computer program include, by way of example, both general and special purpose microprocessors, and anyone or more processors of any kind of digital computer. Generally, a processor receives instructions and data from a read only memory or a random-access memory or both. The essential elements of a computer are a processor for performing instructions and one or more memory devices for storing instructions and data. Generally, a computer also includes, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a GPS receiver, to name just a few. Computer readable media suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The memory may be a non-transitory medium such as a ROM, RAM, flash memory, etc. The processor and the memory can be supplemented by, or incorporated in, special purpose logic circuitry.

The processes and logic flows described in the specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

In accordance with an embodiment, the memory 203 includes both dynamic memory (e.g., RAM, magnetic disk, writable optical disk, etc.) and static memory (e.g., ROM, CD-ROM, etc.) for storing executable instructions that when executed perform the inventive steps described herein to detect and avoid rail way hazards.

In accordance with an embodiment, network includes one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (Wi-Fi), wireless LAN (WLAN), Bluetooth®, Internet Protocol (IP) data casting, ZigBee satellite, mobile ad-hoc network (MANET), and the like, or any combination thereof.

Although the present specification describes components and functions that may be implemented in particular embodiments with reference to particular standards and protocols, the invention is not limited to such standards and protocols. For example, The ZigBee or ZigBee/IEEE 802.15.4 protocol is a specification created for wireless networking. It includes hardware and software standard design for WSN (Wireless sensor network) requiring high reliability, low cost, low power, scalability and low data rate. Accordingly, replacement standards and protocols having the same or similar functions as those disclosed herein are considered equivalents thereof.

In an example embodiment, the communication interface 205 may include but not limited to traditional interfaces which include No intelligence in the interface, only physical connection which could include changes in voltage levels and transformation from balanced to unbalanced signal, communication protocols which may use pre-programmed modules etc. Further, the communication interface may include Modern interfaces, which have a high level of intelligence in the interface where a high level of intelligence in the interface is employed to execute operations.

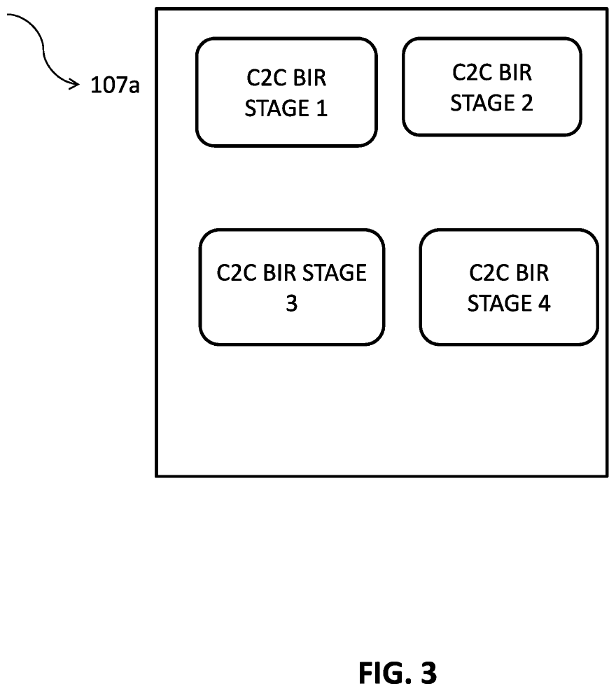

Further, execution of the at least one activity is executed by the system 101 comprising the embedded processor 201, memory 203 and the communication interface 205, based on control and configuration of components associated with the system 101. Functioning of each of the components, to enable advanced neuro rehabilitation, may be observed in the FIG. 3.

FIG. 3 illustrates an example scenario, with a plurality of coma to community (C2C) levels associated with brain injuries for enabling advanced neuro rehabilitation, according to one embodiment of the invention. In operations, the processor 201 is configured to categorize different C2C levels. The categorization is in advanced by administrator based journey or the continuum of patients from coma to community (?? grammar not ok. Not sure how this sentence should read). In some example embodiments, the processor 201 may be configured to categorize the C2C levels based on Artificial intelligence (AI) unit based on the data of past patients' inputs.

In some example embodiments, the staging and level categorization have been arrived through clinical experience and observations spanning over three decades. The level C2C4 describes normal functioning and each level in stages C2C1 to C2C3 describes specific patterns of functional disabilities in patients with brain injuries.

In some example embodiments, the staging and level categorization information is stored in a memory in the server as illustrated in FIG. 3. The categories include the journey or the continuum of patients from coma to community into 4 distinct stages that comprise of 10 levels.

In some example embodiments, the level C2C4 describes normal functioning and each level in stages C2C1 to C2C3 describe specific patterns of functional disabilities in patients with brain injuries. This seems the same as 0051.

In some example embodiments, referring to FIG. 3, C2C BIR stage 1 (called C2C BIR1) is the state when the patient is in altered sensorium/coma (GCS 2T/15 or 3/15 till 14/15). The patient has none or poor awareness of the surroundings. He has non-purposive, purposive and/or involuntary motor, verbal and visual response. He may be dependent on ventilator support or intubated/tracheostomised (more commonly than not). These cases are more commonly encountered in the ICU where they may be brought in the acute phase. Patients in this stage may either progress towards stages 2, 3 or 4 or may end up remaining in a minimal conscious state (MCS) or a persistent vegetative state (PVS). Those who are successful in ‘turning the corner’ with multi-disciplinary and intensive neurorehabilitation may/should gradually regain awareness and progress to C2C stage 2 and beyond.

In some example embodiments, referring to FIG. 3, C2C BIR stage 2 (called C2C BIR2)—Here the patient progresses from a state of basic consciousness (GCS 10T/15 or 14/15), to becoming oriented and aware to all that is happening to him/her internally as well as in the external environment around them and to appropriately respond to these stimuli. Patients in this category are out of coma and are able to process the information from within and external environments. But they are still not aware of ‘what’ and ‘why’, ‘where’ and ‘how’, ‘when’ and ‘how much’. They may have significant physical, cognitive, behavioural, speech, swallowing and functional disabilities. Those on tracheostomy would undergo necessary measures to attempt and close it. These disabilities can be minimized, depending on the anatomical site, the etio-pathology & severity of the injury/insult and the therapy protocols & strategies. They may be in a confused and agitated state and fail to cooperate during the neurorehabilitation program. These patients usually progress further with repetitive and constant push both by the care givers and the rehabilitation professionals, along with ‘tender loving care’. He/she eventually reaches a state of complete awareness, alertness and is oriented to ‘time, place and person’. Such a patient then progresses to C2C stage 3.

In some example embodiments, referring to FIG. 3, C2C stage 3 (called C2C BIR3)—This stage predominantly attempts to restore cognitive, motor planning, programming & execution as well as the speech/language and swallowing capabilities closer to the premorbid levels of functioning. Such patients are able to perform their daily chores with little or no assistance. He/she requires occasional instructions (or supervision) to complete his/her daily schedule, but he/she can be promised that he/she has the potential to return to his/her family and social life. In-depth evaluation may assist the therapist to decide what will be the most suitable destination for these fortunate patients and what level of functionality can be expected of them. By the time they reach the last level of stage 3, they should be able to do some kind of gainful employment.

In some example embodiments, referring to FIG. 3, C2C stage 4 (called C2C BIR4)—These ‘former’ patients have overcome their neurological disabilities and have achieved ‘near normal’ levels of functioning when compared to their premorbid levels. Such individuals constitute the group of people who have successfully completed the entire C2C cycle of rehabilitation—from ‘coma to community’.

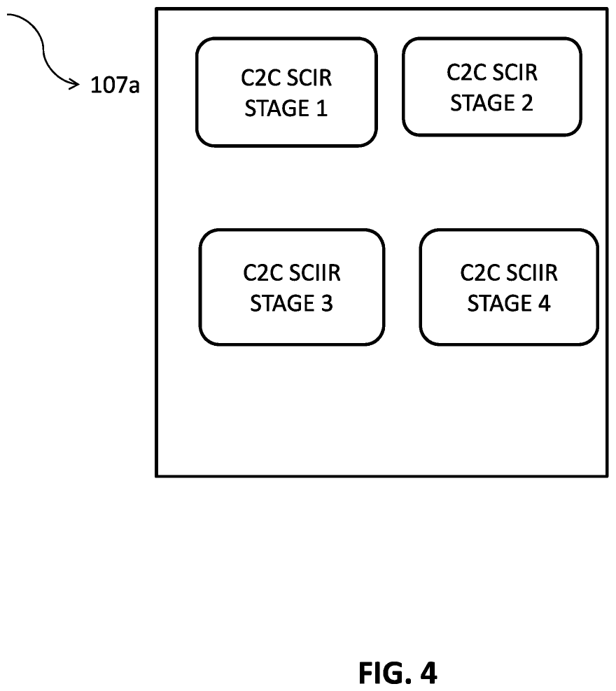

FIG. 4 illustrates an example scenario, with a plurality of cot to community (C2C) for Spinal cord/peripheral nerve Injuries (C2C SCIR) for enabling advanced neuro rehabilitation, according to one embodiment of the invention. In operations, the processor 201 configured to categorize different Spinal cord/peripheral nerve Injuries (C2C SCIR) levels. The categorization is in advanced by administrator/doctor based journey or the continuum of patients from cot to community. In some example embodiments, the processor 201 may be configured to categorize the C2C levels based on Artificial intelligence (AI) unit.

Similar to FIG. 3, the processor 201 may be configured to categorize the journey or the continuum of patients from cot to community into 4 distinct stages that comprise of 10 levels. This staging and level categorization have been arrived through clinical experience and observations spanning over two decades. The level C2C4 describes normal functioning and each level in stages C2C1 to C2C3 describe specific patterns of functional disabilities in patients spinal cord/peripheral nerve injuries—FIG. 4.

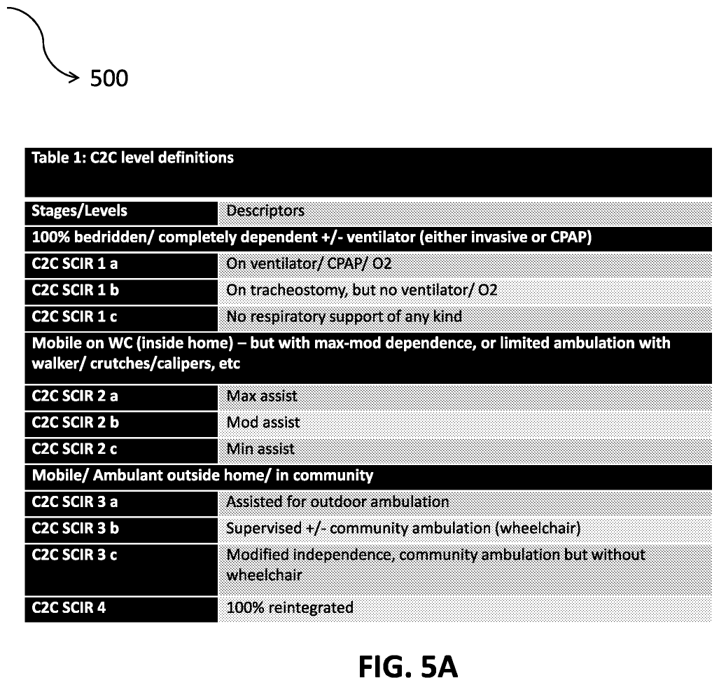

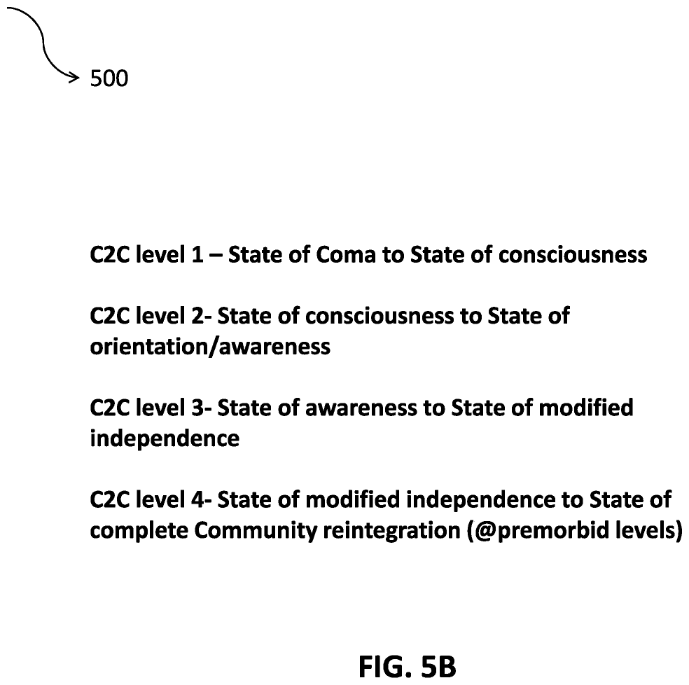

FIGS. 5A and 5B illustrates an example scenario; the NewRo model for rehabilitation of spinal cord/peripheral nerve injuries is called “Cot to Community (C2C)”, according to one embodiment of the invention.

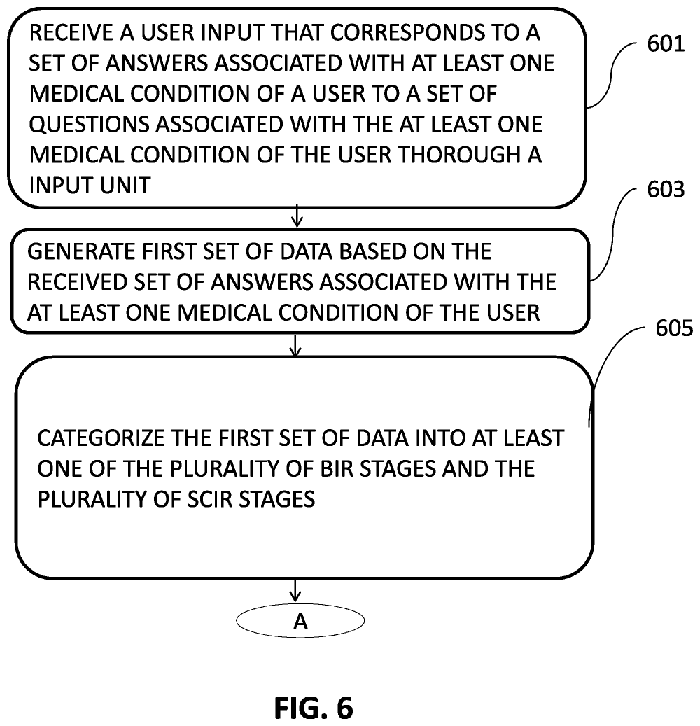

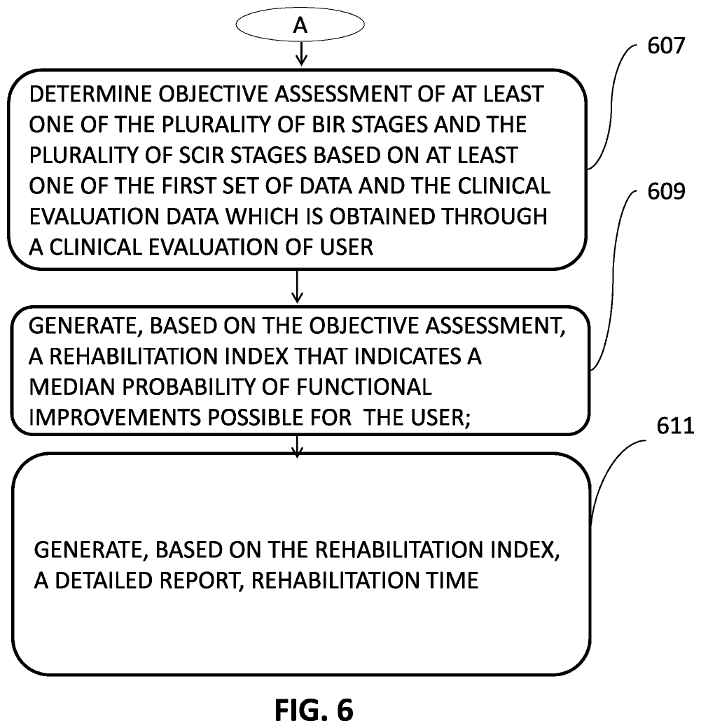

FIG. 6 illustrates an information processing method for enabling advanced neuro rehabilitation, according to one embodiment of the invention. At step 601, the processor 201 is configured to receive a user input that corresponds to a set of answers associated with at least one medical condition of a user to a set of questions associated with the at least one medical condition of the user thorough a input unit.

The set of questions may be referred as Self-Evaluation of Functional Abilities (SEFA®). The SEFA is designed to gather in a layman's language, the patient-reported details of functional disability related to brain and spinal cord/peripheral nerve injuries to arrive at irC2C (the baseline condition of a patient). The irC2C is an objective description of a patient's condition. The SEFA has a series of over 700 distinct responses/questions that are categorised under many different and distinct headings, called subsections. On an average, the person undertaking SEFA test has to answer between 75-110 questions, following which they get a pre-written standardised SEFA report.

In some example embodiments, at step 603, the processor 201 is configured to generate first set of data based on the received set of answers associated with the at least one medical condition of the user. The first set of data corresponds to mapping of each answer to a particular C2C level (either BIR or SCIR-10 levels each).

In some example embodiments, at step 605, the processor 201 is configured to categorize the first set of data into at least one of the plurality of BIR stages and the plurality of SCIR stages. The categorization is explained in detail with respect to FIG. 3 and FIG. 4.

This processor is configured to enable a patient/user with injuries to the brain, spinal cord or it's extensions into 10 distinct ‘clinical’ buckets, irrespective of what kind of injury the patient sustained (i.e.—stroke, traumatic brain injury, traumatic spinal cord injury, spinal cord tumors, GB syndrome, etc). The system focuses primarily on the ‘functioning abilities/disabilities’ of the patients as the primary and only metric of defining these levels. The categorization is explained in detail with respect to FIG. 3 and FIG. 4.

In some example embodiments, at step 607, the processor 201 is configured to determine objective assessment of at least one of the plurality of BIR stages and the plurality of SCIR stages based on at least one of the first set of data and the clinical evaluation data which is obtained through a clinical evaluation of user. Pre-written SEFA reports have about 75-90% accuracy to that patients' functional condition.

In some example embodiments, the objective assessment that corresponds to Clinical Evaluation of Functional Abilities (CEFA®). It is a way that the clinicians (neurologists, neurosurgeons, rehab physicians or rehab professionals like therapists and nurses) are able to assess the C2C levels of the patient and match it with the SEFA. Referring to FIG. 1, the clinical evaluation apparatus may be configured to collect and evaluate data. The clinical evaluation apparatus involves a complex analysis of data acquired by using certain internationally accepted evaluation scores, a MRI/CT scanner, X-ray detector or any other device that detect particular injuries/disabilities of the brain and spinal cord.

The Rationale is since the C2C level concept itself has been proposed and used only by us, a combination of SEFA and CEFA helps us to test the stability of this complex idea as well as validate the robustness of the product across multiple use cases and multiple clinical scenarios. This is agnostic to region, race, sex, background, etc of the patient.

The calculations of C2C levels in done in a way where SEFA uses data obtained from simple day-to-day movements and activities of the patient to arrive at the C2C level of that patient. This is done using a complex and innovative logic. Now CEFA on the other hand uses (the data) from 4 internationally accepted, standardised scales that are measuring consciousness, disability, cognitive functions and disability in day-to-day tasks. Here again we have created a unique matrix that uses the various data from these scales. This complex and innovative logic helps us to again arrive at the C2C level—but from a very different perspective. As described in above, in spite of using totally different concepts and data points, one can able to arrive at the same C2C scores. This ‘double verification’ has made our product even more stable and robust.

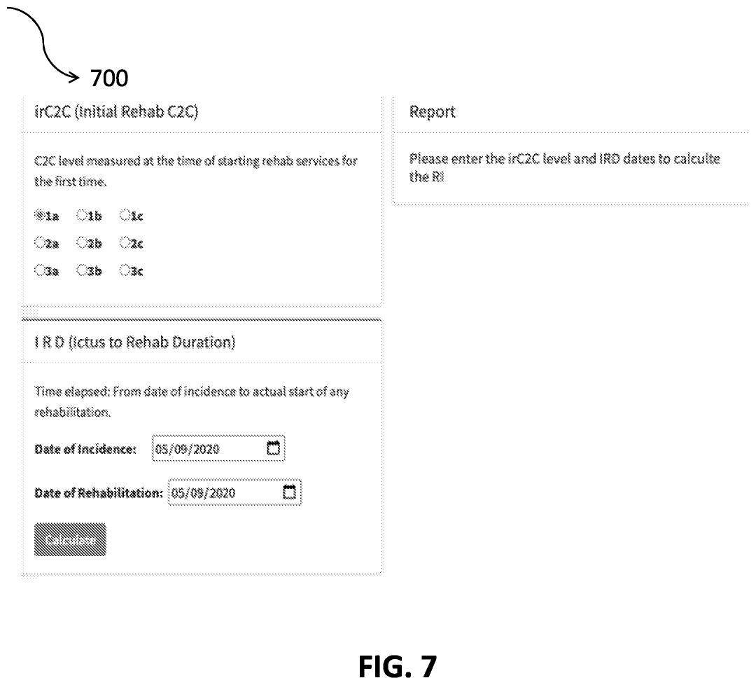

FIG. 7 illustrates generating a rehabilitation index (RI) for enabling advanced neuro rehabilitation, according to one embodiment of the invention. In some example embodiments, at step 609, the processor 201 is configured to generate, based on the objective assessment, a rehabilitation index (RI) that indicates a median probability of the optimal functional improvements possible for the user.

Before treatment initiation, patients want to know the odds of treatment success. Clinicians have to be sure-footed with probabilities of good outcomes. RI does this work by indicating the median probability of functional improvements possible for a given patient. Using inputs from patients through the SEFA® (Self Evaluation of functional abilities) questionnaire and CEFA® (Clinical evaluation of functional abilities), duration between onset of injury to the rehab consultation is used to compute the RI. The RI helps in predicting the prognosis, mapping the progress and planning the process of optimal rehabilitation strategies to achieve “coma-to-community” and “cot-to-community” end points.

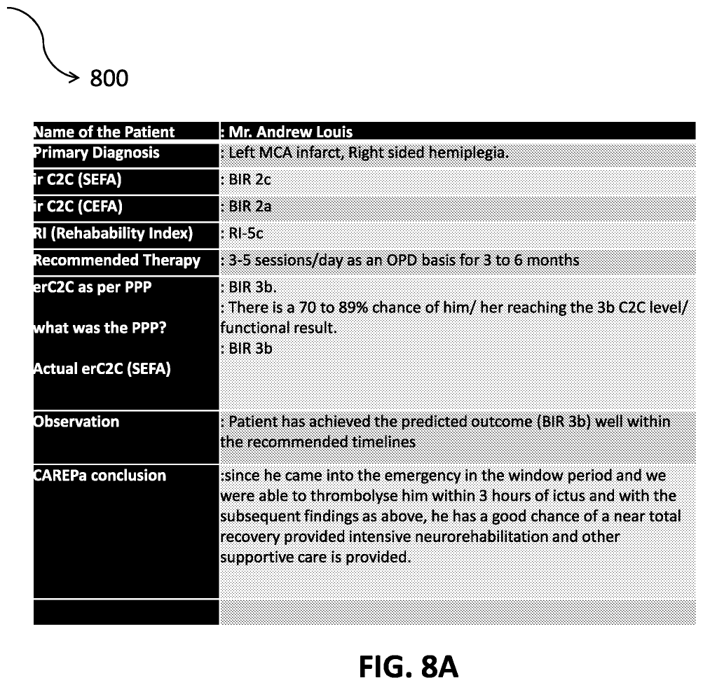

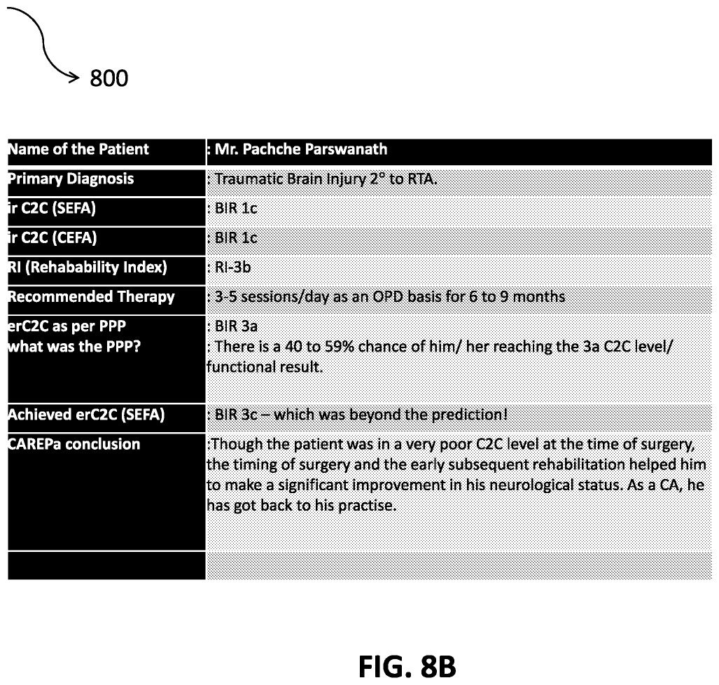

FIGS. 8A and 8B illustrate an example scenario, showing case example based on the C2C levels associated with brain & spinal cord injuries for enabling advanced neuro rehabilitation, according to one embodiment of the invention. In some example embodiments, at step 611, the processor 201 is configured to generate, based on the rehabilitation index, a detailed report that indicates the type of diagnosis, time of diagnosis, rehabilitation time, and the specific description of the diagnosis.

The detailed report then analysed based on novel treatment protocols are guided by a novel principle called the CAREPa-Re principle. CAREPa-Re (read care-pa-re) is an acronym for Clinical, Anatomical, Radiological, Etio-Pathological and Rehabilitation. The system may configured to map the dynamics of CAREPa with Rehabilitation and have defined the CAREPa-Re principle, which helps in understanding the areas of the brain affected and hence the brain circuits that could be damaged is critical for prognosticating the future progress and hence to predicting eventual ‘functional’ outcomes.

By dynamically tracking the initial CAREPa to the subsequent CAREPa findings, the predictions could change (for the better or sometimes even for the worse). As the patient improves, the CAREPa will help anticipate and uncover more advanced yet subtle and complex neurological disabilities (like apraxias, agnosias, visuo-spatial issues, etc) all of which can impact the PPP.

The CAREPa tree is designed to effectively capture relevant data as it captures the initial clinical findings at ictus, the subsequent worsening due to the progress of the disease, related and unrelated complications (like raised ICP, brain surgery, electrolyte disturbances, effective control of hypertension and diabetes, infections, etc), speed of improvement, areas of the brain affected, the type of etio-pathology, etc.

This system is the first ever attempt to qualify a patient with injuries to the brain, spinal cord or it's extensions into 10 distinct ‘clinical’ buckets, irrespective of what kind of injury the patient sustained (i.e.—stroke, traumatic brain injury, traumatic spinal cord injury, spinal cord tumors, GB syndrome, etc). This system focuses primarily on the ‘functioning abilities/disabilities’ of the patients as the primary and only metric as the means of defining these levels. This whole para has been said earlier and could be deleted.

The invention made a fundamental shift in the way the clinicians analyse and categorise the patients—from the current perspective of ‘individual diseases, their severity, the temporal profile & complications’ to simply a ‘functional abilities’ based scaling and scoring.

The invention provides the rehab professionals with a crystal-clear path and plan of action which can then be easily explained to the patient and his/her caregivers (by relating it to their day-to-day lives and not some complicated medical jargons)—hence simplifying the communications and also reducing the stress.—this whole para has been said earlier and could be deleted if necessary.

It also helps the three stakeholders—the patient, his/her caregivers and the rehab professionals to come up with mutually agreed upon goals that will motivate the patients to work. This will also help the rehab professionals to create ‘customised treatment plans’ which may best help in achieving these outcomes.

Finally, the invention provides a structured plan for achieving the predicted prognosis.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications within the spirit and scope of the embodiments as described herein.

We claim:

1. A system (100) for advanced neuro rehabilitation, comprising:
a memory (107a) configured to store at least one of a plurality of Brain Injury Rehabilitation (BIR) stages and a plurality of Spinal cord Injury Rehabilitation (SCIR) stages; and
circuitry (201) configured to:
receive a user input that corresponds to a set of answers associated with at least one medical condition of a user to a set of questions associated with the at least one medical condition of the user thorough a input unit;
generate first set of data based on the received set of answers associated with the at least one medical condition of the user; and
categorize the first set of data into at least one of the plurality of BIR stages and the plurality of SCIR stages, wherein the first set of data comprises a plurality of data sets under specific categories coded for both BIR & SCIR.
determine objective assessment of at least one of the plurality of BIR stages and the plurality of SCIR stages based on the plurality of data sets and combined analysis using evidence-based scientific reasoning.
generate, based on the objective assessment, a rehabilitation index that indicates a median probability of functional improvements possible for the user; and
generate, based on the rehabilitation index, a detailed report that indicates the type of diagnosis, time of diagnosis, rehabilitation time, C2C scores, and predictability of reaching the outcome C2C score.

2. The system as claimed in claim 1, wherein the plurality of BIR & SCIR stages include C2C BIR stages 1-4 and C2C SCIR stages 1-4.

3. The system as claimed in claim 2, wherein the plurality of BIR & SCIR stages includes at least four stages of both BIR & SCIR and at least ten levels each of both BIR & SCIR.

4. The system as claimed in claim 1, wherein the C2C BIR stage 1 is corresponds to:
the state of the user when the user is in altered sensorium/coma, and
a patient whose condition corresponds a 100% bedridden completely dependent or support of ventilator (either invasive or CPAP).

5. The system as claimed in claim 1, wherein the C2C BIR stage 2 correspond to:
the state of the user in which the user progresses from a state of basic consciousness (GCS 10T/15 or 14/15), to becoming oriented and aware to all that is happening to the user internally as well as in the external environment around the user and to appropriately respond to a specific stimuli, and
a patient whose condition corresponds to mobile on wheel chair but with maximum/moderate dependence, or limited ambulation with walker or crutches or calipers.

6. The system as claimed in claim 1, wherein the C2C stage 3 correspond to:
a stage in which the user predominantly attempts to restore cognitive, motor planning, programming & execution as well as the speech/language and swallowing capabilities closer to the premorbid levels of functioning, and
a patient whose condition corresponds to being mobile or ambulant outside home and in the community.

7. The system as claimed in claim 1, wherein the C2C stage 4 correspond to:
a state in which the user have overcome the neurological disabilities and have achieved normal levels of functioning when compared to their premorbid levels, and
a patient whose condition corresponds to 100% reintegration into the community.

8. The system as claimed in claim 1, wherein the set of questions and set of answers are in any specific language.

9. The system as claimed in claim 1, wherein the user input configured to receive input from input via input unit based on written answers of the user or voice/video input of the user.

10. An information processing method (600) for advanced neuro rehabilitation, comprising:
receiving a user input that corresponds to a set of answers associated with at least one medical condition of a user to a plurality of sets of categorised questions associated with the at least one medical condition of the user thorough a input unit;
generating first set of data based on the received set of answers associated with the at least one medical condition of the user; and
categorizing the first set of data into at least one of the plurality of BIR stages and the plurality of SCIR stages;
determining objective assessment of at least one of the plurality of BIR stages and the plurality of SCIR stages based on at the coded output ‘logic’ of the plurality of categorised sets of questions and the clinical evaluation data which is obtained through a clinical evaluation of user;
generating, based on the objective assessment, a rehabilitation index that indicates a median probability of functional improvements possible for the user; and
generating, based on the rehabilitation index (RI), a detailed report that indicates the type of diagnosis, time of diagnosis, rehabilitation time, and the specific description of the diagnosis.
